(19) United States
(12) Patent Application Publication
(10) Pub. No.: US 2010/0022390 A1
(43) Pub. Date: Jan. 28, 2010

(54) RESISTANCE MANAGEMENT STRATEGY

(75) Inventor: Daniel J. Cosgrove, Johnston, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., JOHNSTON, IA (US)

(21) Appl. No.: 12/520,758

(22) PCT Filed: Dec. 26, 2007
(86) PCT No.: PCT/US07/88825

§ 371 Date: Jun. 22, 2009

Related U.S. Application Data
(60) Provisional application No. 60/871,671, filed on Dec. 22, 2006.

(51) Int. Cl. A01N 63/00; A01H 5/00; A01N 57/00

(52) U.S. Cl. 504/118; 800/302; 514/75

(57) ABSTRACT

A method for managing resistance in a plot of pest resistant crop plants is provided herein. The method includes cultivating a first pest resistant crop plant in a plot in one planting cycle, and successively cultivating in a second planting cycle a second pest resistant crop plant in the same plot, wherein the first and the second pest resistant crop plants are pesticidal to corn rootworm but through a different mode of pesticidal action.

### REFERENCE TO RELATED APPLICATIONS

This application claims priority to U.S. Provisional Application Ser. No. 60/871,671, filed Dec. 22, 2006, the contents of which are incorporated by reference in their entirety.

### FIELD OF THE INVENTION

The present invention relates to methods for managing resistance in a plot of pest resistant crop plants.

### BACKGROUND OF THE INVENTION

Insects, nematodes, and related arthropods annually destroy an estimated 15% of agricultural crops in the United States and even more than that in developing countries. Yearly, these pests cause over 100 billion dollars in crop damage in the U.S. alone. In addition, competition with weeds and parasitic and saprophytic plants account for even more potential yield losses.

Some of this damage occurs in the soil when plant pathogens, insects and other such soil borne pests attack the seed after planting. In the production of corn, for example, much of the damage is caused by rootworms, insect pests that feed upon or otherwise damage the plant roots, and by cutworms, European corn borers, and other pests that feed upon or damage the above ground parts of the plant. General descriptions of the type and mechanisms of attack of pests on agricultural crops are provided by, e.g., Metcalf (1962), in Destructive and Useful Insects, 4th ed. (McGraw-Hill Book Co., NY); and Agrios (1988), in Plant Pathology, 3d ed. (Academic Press, NY).

In an ongoing seasonal battle, farmers must apply billions of gallons of synthetic pesticides to combat these pests. However, synthetic pesticides pose many problems. They are expensive, costing U.S. farmers alone almost 8 billion dollars per year. They force the emergence of insecticide-resistant pests, and they can harm the environment.

Because of concern about the impact of pesticides on public health and the health of the environment, significant efforts have been made to find ways to reduce the amount of chemical pesticides that are used. Recently, much of this effort has focused on the development of transgenic crops that are engineered to express insect toxicants derived from microorganisms. For example, U.S. Pat. No. 5,877,012 to Estruch et al. discloses the cloning and expression of proteins from such organisms as Bacillus, Pseudomonas, Clavibacter and Rhizobium into plants to obtain transgenic plants with resistance to such pests as black cutworms, armyworms, several borers and other insect pests. Publication WO/EP97/07089 by Privalle et al. teaches the transformation of monocotyledons, such as corn, with a recombinant DNA sequence encoding peroxidase for the protection of the plant from feeding by corn borers, earworms and cutworms. Jansens et al. (1997) Crop Sci., 37(5): 1616-1624, reported the production of transgenic corn containing a gene encoding a crystalline protein from Bt that controlled both generations of European corn borer (ECB). U.S. Pat. Nos. 5,625,136 and 5,859,336 to Koziel et al reported that the transformation of corn with a gene from Bt that encoded for a δ-endotoxin provided the transgenic corn with improved resistance to ECB. A comprehensive report of field trials of transgenic corn that expresses an insecticidal protein from Bacillus thuringiensis (Bt) has been provided by Armstrong et al., in Crop Science, 35(2):550-557 (1995).

An environmentally friendly approach to controlling pests is the use of pesticidal crystal proteins derived from the soil bacterium Bacillus thuringiensis (Bt), commonly referred to as “Cry proteins” or “Cry peptides.” The Cry proteins are globular protein molecules which accumulate as protoxins in crystalline form during late stage of the sporulation of Bt. After ingestion by the pest, the crystals are solubilized to release protoxins in the alkaline midgut environment of the larvae. Protoxins (˜130 kDa) are converted into toxic fragments (˜66 kDa N terminal region) by gut proteases. Many of these proteins are quite toxic to specific target insects, but harmless to plants and other non-targeted organisms. Some Cry proteins have been recombinantly expressed in crop plants to provide pest-resistant transgenic plants. Among those, Bt-transgenic cotton and corn have been widely cultivated.

A large number of Cry proteins have been isolated, characterized and classified based on amino acid sequence homology (Crickmore et al., 1998, Microbiol. Mol. Biol. Rev., 62: 807-813). This classification scheme provides a systematic mechanism for naming and categorizing newly discovered Cry proteins. The Cry1 classification is the best known and contains the highest number of cry genes which currently totals over 130.

One biotype of western corn rootworm (WCRW), which deposits its eggs in soybeans and possibly other crop habitats, is now capable of causing significant injury to first-year corn (i.e., corn that has not systematically followed corn). This biotype is commonly called first-year corn rootworm or rotation-resistant corn rootworm. First-year corn may also be susceptible to rootworm injury when eggs remain in the soil for more than a year. In this situation, the eggs deposited in the plot remain dormant throughout the following year and then hatch the next year, when corn may again be planted in a two-year rotation cycle. Such rootworm activity is called extended diapause and is commonly associated with northern corn rootworm (NCRW), especially in the northwestern region of the Corn Belt.

Further, most countries, including the United States, require implementation of an insect resistance management (IRM) plan when transgenic crops are commercialized in order to minimize the development of resistant pests, and thereby extend the useful life of known biopesticides. One of the most common components of an IRM plan is a refuge is where in a given crop, 80% of the seed planted may contain a transgenic event which kills a target pest (such as WCRW), but 20% of the seed must not contain a transgenic event with activity against the target pest. The goal of such a refuge strategy is to prevent the target pests from developing resistance to the particular biopesticide produced by the transgenic crop. Because those target insects that reach maturity in the 80% transgenic area could be more likely to carry resistance genes to the biopesticide used there, the refuge permits adult WCRW insects to develop that are not resistant to the biopesticide used in the transgenic seeds. As a result, the non-resistant insects breed with the resistant insects, and, because the resistance gene is typically recessive, eliminate much of the resistance in the next generation of insects. The problem with this refuge strategy is that in order to produce susceptible insects, some of the crop planted must be susceptible to the pest, thereby reducing yield.

As indicated above, one concern is that resistant ECB, WCRW, or other pests will emerge. One strategy for combating the development of resistance is to select a recombinant corn event which expresses high levels of the insecticidal protein such that one or a few bites of a transgenic corn plant would cause at least total cessation of feeding and subsequent death of the pest, even if the pest is heterozygotic for the resistance trait (i.e., the pest is the result of a resistant pest mating with a non-resistant pest).

Another strategy would be to combine a second ECB or WCRW specific insecticidal protein in the form of a recombinant event in the same plant or in an adjacent plant, for example, another Cry protein or alternatively another insecticidal protein such as a recombinant acyl lipid hydrolase or insecticidal variant thereof. See, e.g., WO 01/49834. Preferably, the second toxin or toxin complex would have a different mode of action than the first toxin, and preferably, if receptors were involved in the toxicity of the insect to the recombinant protein, the receptors for each of the two or more insecticidal proteins in the same plant or an adjacent plant would be different so that if a change of function of a receptor or a loss of function of a receptor developed as the cause of resistance to the particular insecticidal protein, then it should not and likely would not affect the insecticidal activity of the remaining toxin which would be shown to bind to a receptor different from the receptor causing the loss of function of one of the two insecticidal proteins cloned into a plant. Accordingly, the first one or more transgenes and the second one or more transgenes are preferably insecticidal to the same target insect and bind without competition to different binding sites in the gut membranes of the target insect.

Still another strategy would combine a chemical pesticide with a pesticidal protein expressed in a transgenic plant. This could conceivably take the form of a chemical seed treatment of a recombinant seed which would allow for the dispersal into a zone around the root of a pesticidally controlling amount of a chemical pesticide which would protect root tissues from target pest infestation so long as the chemical persisted or the root tissue remained within the zone of pesticide dispersed into the soil.

Another alternative to the conventional forms of pesticide application is the treatment of plant seeds with pesticides. The use of fungicides or nematicides to protect seeds, young roots, and shoots from attack after planting and sprouting, and the use of low levels of insecticides for the protection of, for example, corn seed from wireworm, has been used for some time. Seed treatment with pesticides has the advantage of providing for the protection of the seeds, while minimizing the amount of pesticide required and limiting the amount of contact with the pesticide and the number of different field applications necessary to attain control of the pests in the field.

Other examples of the control of pests by applying insecticides directly to plant seed are provided in, for example, U.S. Pat. No. 5,696,144, which discloses that ECB caused less feeding damage to corn plants grown from seed treated with a 1-arylpyrazole compound at a rate of 500 g per quintal of seed than control plants grown from untreated seed. In addition, U.S. Pat. No. 5,876,739 to Turnblad et al. (and its parent, U.S. Pat. No. 5,849,320) disclose a method for controlling soil-borne insects which involves treating seeds with a coating containing one or more polymeric binders and an insecticide. This reference provides a list of insecticides that it identifies as candidates for use in this coating and also names a number of potential target insects.

Although recent developments in genetic engineering of plants have improved the ability to protect plants from pests without using chemical pesticides, and while such techniques such as the treatment of seeds with pesticides have reduced the harmful effects of pesticides on the environment, numerous problems remain that limit the successful application of these methods under actual field conditions.

Insect resistance management (IRM) is the term used to describe practices aimed at reducing the potential for insect pests to become resistant to a pesticide. Maintenance of Bt IRM is of great importance because of the threat insect resistance poses to the future use of Bt plant-incorporated protectants and Bt technology as a whole. Specific IRM strategies, such as the high dose/structured refuge strategy, mitigate insect resistance to specific Bt proteins produced in corn, cotton, and potatoes. However, such strategies result in portions of crops being left susceptible to one or more pests in order to ensure that non-resistant insects develop and become available to mate with any resistant pests produced in protected crops. Accordingly, from a farmer/producer's perspective, it is highly desirable to have as small a refuge as possible and yet still manage insect resistance, in order that the greatest yield be obtained while still maintaining the efficacy of the pest control method used, whether Bt, chemical, some other method, or combinations thereof.

The most frequently-used current IRM strategy is a high dose and the planting of a refuge (a portion of the total acreage using non-Bt seed), as it is commonly-believed that this will delay the development of insect resistance to Bt crops by maintaining insect susceptibility. The high dose/refuge strategy assumes that resistance to Bt is recessive and is conferred by a single locus with two alleles resulting in three genotypes: susceptible homozygotes (SS), heterozygotes (RS), and resistant homozygotes (RR). It also assumes that there will be a low initial resistance allele frequency and that there will be extensive random mating between resistant and susceptible adults. Under ideal circumstances, only rare RR individuals will survive a high dose produced by the Bt crop. Both SS and RS individuals will be susceptible to the Bt toxin. A structured refuge is a non-Bt portion of a grower's field or set of fields that provides for the production of susceptible (SS) insects that may randomly mate with rare resistant (RR) insects surviving the Bt crop to produce susceptible RS heterozygotes that will be killed by the Bt crop. This will remove resistant (R) alleles from the insect populations and delay the evolution of resistance. MON810, BT11, and TC1507 are currently-available products believed to be “high dose” against ECB.

The high dose/refuge strategy is the currently-preferred strategy for IRM. Non-high dose strategies are currently used in an IRM strategy by increasing refuge size. The refuge is increased because lack of a high dose could allow partially resistant (i.e., heterozygous insects with one resistance allele) to survive, thus increasing the frequency of resistance genes in an insect population. For this reason, numerous IRM researchers and expert groups have concurred that non-high dose Bt expression presents a substantial resistance risk relative to high dose expression (Roush 1994, Gould 1998, Onstad & Gould 1998, SAP 1998, ILSI 1998, UCS 1998, SAP 2001). However, such non-high dose strategies are typically unacceptable for the farmer, as the greater refuge size results in further loss of yield.

Currently, the size, placement, and management of the refuge is considered critical to the success of the high dose/structured refuge strategy to mitigate insect resistance to the Bt proteins produced in corn, cotton, and potatoes. Structured refuges are generally required to include all suitable non-Bt host plants for a targeted pest that are planted and managed by people. These refuges could be planted to offer refuges at the same time when the Bt crops are available to the pests or at times when the Bt crops are not available. The problems with these types of refuges include ensuring compliance with the requirements by individual farmers. Because of the decrease in yield in refuge planting areas, some farmers choose to eschew the refuge requirements, and others do not follow the size and/or placement requirements. These non-compliance issues result in either no refuge or less effective refuge, and a corresponding increase in the development of resistance pests.

### European Corn Borer (ECB)

ECB is a major pest of corn throughout most of the United States. The pest has 1-4 generations per year, with univoltine (i.e., one generation per year) populations in the far North (i.e., all of North Dakota, northern South Dakota, northern Minnesota, and northern Wisconsin), bivoltine (i.e., two generations per year) populations throughout most of the Corn Belt, and multivoltine (3-4 generations) populations in the South (Mason et al. 1996). A summary of key aspects of ECB biology that relate to IRM is presented below:

### Larval Movement

ECB larvae are capable of significant, plant-to-plant movement within corn fields. Research conducted in non-transgenic corn showed that the vast majority of larvae do not move more than two plants within a row (Ross & Ostlie 1990). However, in transgenic corn, unpublished data (used in modeling work) from F. Gould (cited in Onstad & Gould 1998) indicates that approximately 98% of susceptible ECB neonates move away from plants containing Bt. Recent multi-year studies by Hellmich (1996, 1997, 1998) have attempted to quantify the extent of plant-to-plant larval movement. It was observed that 4th instar larvae were capable of movement up to six corn plants within a row and six corn plants across rows from a release point. Movement within a row was much more likely than movement across rows (not surprising, due to the fact that plants within are row are more likely to be “touching” as opposed to those across rows). In fact, the vast majority of across row movement was limited to one plant. This type of information has obvious implications for optimal refuge design. Larvae moving across Bt and non-Bt corn rows may be exposed to sublethal doses of protein, increasing the likelihood of resistance (Mallet & Porter 1992). Given the extent of ECB larval movement between plants, prevailing belief is that seed mixes are an inferior refuge option (Mallet & Porter 1992, SAP 1998, Onstad & Gould 1998).

### Adult Movement

Information on movement of adult ECB (post-pupal eclosion) is necessary to determine appropriate proximity guidelines for refuges. Refuges must be established within the flight range of newly emerged adults to help ensure the potential for random mating. An extensive, multi-year project to investigate ECB adult dispersal was undertaken by the University of Nebraska (Hunt et al. 1997, 1998a). Results from these mark and recapture studies (with newly emerged, pre-mated adults) showed that the majority of ECB adults did not disperse far from their emergence sites. The percentage recaptured was very low (<1%) and the majority of those that were recaptured were caught within 1500 feet of the release site. Few moths were captured outside of 2000 feet. These results have specifically led to recommendations and guidelines for refuge proximity and deployment.

### Mating Behavior

In addition to patterns of adult movement, ECB mating behavior is an important consideration to insure random mating between susceptible and potentially resistant moths. In particular, it is important to determine where newly emerged females mate (i.e., near the site of emergence or after some dispersal). It is well established that many ECB take advantage of aggregation sites (usually clusters of weeds or grasses) near corn fields for mating. Females typically mate the second night after pupal eclosion (Mason et al. 1996). One recent study suggested that it may be possible to manipulate aggregation sites to increase the likelihood of random mating between susceptible and potentially resistant ECB (Hellmich et al. 1998). Another recent study (mark/recapture studies with newly eclosed ECB) conducted by the University of Nebraska showed that relatively few unmated females moved out of the corn field from which they emerged as adults (Hunt et al. 1998b). This was especially true in irrigated (i.e., attractive) corn fields. In addition, a relatively high proportion of females captured close to the release point (within 10 feet) were mated. This work suggests that females mate very close to the point of emergence and that refuges may need to be placed very close to Bt fields (or as in-field refuges) to maximize the probability of random mating.

In terms of male mating behavior, a study by Showers et al. (2001) looked at male dispersal to locate mates. The study was carried out using mark-recapture techniques with pheromone-baited traps placed at 200, 800, 3200, and 6400 m from a release point. Results showed that males in search of mates were trapped more frequently at traps placed at 200 m from the release site. However, significant numbers were also trapped at 800 m or greater from the release site (Showers et al. 2001). Similar to Hunt et al., this work suggests that refuges may need to be placed relatively close to Bt fields to maximize random mating.

### Ovipositional Behavior

ECB ovipositional (egg-laying) behavior is also important for refuge design. For instance, if oviposition within a corn field is not random, certain types of refuge (i.e., in-field strips) may not be effective. After mating, which occurs primarily in aggregation sites, females move to find suitable corn hosts for oviposition. Most females will oviposit in corn fields near the aggregation sites, provided there are acceptable corn hosts. Oviposition begins after mating and occurs primarily at night. Eggs are laid in clusters of up to sixty eggs (one or more clusters are deposited per night) (Mason et al. 1996).

It is known that females generally prefer taller and more vigorous corn fields for oviposition (Beck 1987). This has implications for refuge design. To avoid potential host discrimination among ovipositing females, the non-Bt corn hybrid selected for refuge should similar to the Bt hybrid in terms of growth, maturity, yield, and management practices (i.e., planting date, weed management, and irrigation). It should be noted that research has shown no significant difference in ovipositional preferences between Bt and non-Bt corn (derived from the same inbred line) when phenological and management characteristics are similar (Orr & Landis 1997, Hellmich et al. 1999). Within a corn field suitable for egg laying, oviposition is thought to be random and not restricted to border rows near aggregation sites (Shelton et al. 1986, Calvin 1998).

### Host Range

ECB is a polyphagous pest known to infest over 200 species of plants. Among the ECB plant hosts are a number of species of common weeds, which has led some to speculate that it may be possible for weeds to serve as an ECB refuge for Bt corn, a concept commonly referred to as “unstructured refuge.” In response to this, a number of recent research projects have investigated the feasibility of weeds as refuge. Studies conducted by Hellmich (1996, 1997, 1998) have shown that weeds are capable of producing ECB, although the numbers were variable and too inconsistent to be a reliable source of ECB refuge. This conclusion was also reached by the 1998 SAP Subpanel on IRM. In addition to weeds, a number of grain crops (e.g., wheat, sorghum, oats) have been investigated for potential as a Bt corn ECB refuge (Hellmich 1996, 1997, 1998, Mason et al. 1998). In these studies, small grain crops generally produced less ECB than corn (popcorn or field corn) and were therefore considered unlikely to produce enough susceptible adult insects to be an acceptable refuge. Therefore, based on the current state of the art, an improved IRM for ECB is needed.

### Corn Earworm (CEW)

As with ECB, the 1998 SAP identified a number of research areas that need additional work with CEW. In addition to increased knowledge regarding larval/adult movement, mating behavior, and ovipositional behavior, a better understanding of movement between corn/cotton and long distance migration is also needed (SAP 1998). Additional research regarding CEW biology has occurred since 1998. These data have been submitted as part of the annual research reports required as a condition of registration of such Bt crops before commercial use is permitted. The Agency has reviewed these data and has concluded that additional information would be useful for effective long-term improvements of IRM strategies to mitigate CEW resistance.

### Host Range and Corn to Cotton Movement

CEW is a polyphagous insect (3-4 generations per year), feeding on a number of grain and vegetable crops in addition to weeds and other wild hosts. Typically, it is thought that CEW feeds on wild hosts and/or corn for two generations (first generation on whorl stage corn, second generation on ear stage corn). After corn senescence, CEW moves to other hosts, notably cotton, for 2-3 additional generations. By utilizing multiple hosts within the same growing season, CEW presents a challenge to Bt resistance management in that there is the potential for double exposure to Bt protein in both Bt corn and Bt cotton (potentially up to five generations of exposure in some regions).

### Overwintering Behavior

CEW are known to overwinter in the pupal stage. Although it is known that CEW migrate northward during the growing season to corn-growing regions (i.e., the U.S. Corn Belt and Canada), CEW typically are not capable of overwintering in these regions. Rather, CEW are known to overwinter in the South, often in cotton fields. Temperature, moisture, and cultivation practices are all thought to play some role in the overwintering survival of CEW (Caprio & Benedict 1996).

Overwintering is an important consideration for IRM-resistant insects must survive the winter to pass their resistance genes on to future generations. In the Corn Belt, for example, CEW incapable of overwintering should not pose a resistance threat. Given that different refuge strategies may be developed based upon where CEW is a resistance threat, accurate sampling data would help to precisely predict suitable CEW overwintering areas.

### Adult Movement and Migration

CEW is known to be a highly mobile pest, capable of significant long distance movement. Mark/recapture studies have shown that CEW moths are capable of dispersing distances ranging from 0.5 km (0.3 mi.) to 160 km (99 mi.); some migration up to 750 km (466 mi.) was also noted (Caprio & Benedict 1996). The general pattern of migration is a northward movement, following prevailing wind patterns, with moths originating in southern overwintering sites moving to corn-growing regions in the northern U.S. and Canada.

It has been assumed that CEW migration proceeds progressively northward through the course of the growing season. However, observations made by Dr. Fred Gould (N.C. State University) indicate that CEW may also move southward from corn-growing regions back to cotton regions in the South (described in remarks made at the 1999 EPA/USDA Workshop on Bt Crop Resistance Management in Cotton, Memphis, Tenn. 8/26/99). If this is true, the result may be additional CEW exposure to Bt crops. In addition, the assumptions regarding CEW overwintering may need to be revisited—moths that were thought to be incapable of winter survival (and thus not a resistance threat) may indeed be moving south to suitable overwintering sites.

Most CEW flight movement is local, rather than migratory. Heliothine moths move primarily at night, with post-eclosion moths typically flying short distances of less than 200 m (Caprio & Benedict 1996). However, as was indicated by the 1998 SAP, additional research would be useful, particularly as it pertains to CEW and optimal refuge design. On the other hand, given the long distance movements typical of CEW and the lack of high dose in Bt corn hybrids, the 2000 SAP noted that refuge placement for this pest is of less importance than with other pests (e.g., ECB) (SAP 2001).

### Mating/Ovipositional Behavior

Dr. Michael Caprio (entomologist, Mississippi State University) has indicated that there is significant localized mating among females (i.e., within 600 m (1969 ft.) of pupal eclosion), typically with males that emerged nearby or moved in prior to female eclosion (Caprio 1999). CEW females typically deposit eggs singly on hosts. A recent study (conducted in cotton fields) found that 20% of the eggs found from released CEW females were within 50-100 m (164-328 ft.) of the release point, indicating some localized oviposition. However, males were shown to be able to move over 350 m (1148 ft.) to mate with females (Caprio 2000). These data indicate that, in terms of CEW, refuges may not have to be embedded or immediately adjacent to a Bt field to be effective (although the data do not exclude these options). Additional research with mating and ovipositional behavior would provide useful information for CEW IRM.

### Larval Movement

CEW larvae, particularly later instars, are capable of plant-to-plant movement. At the recommendation of the SAP (1998), the EPA eliminated seed mixes as a viable refuge option for CEW for Bt cotton. Accordingly, an improved IRM strategy for CEW is also needed.

### Southwestern Corn Borer

Some SWCB pest biology data have been provided to the EPA as part of the annual research reports required as a condition of registration. However, there is still relatively limited information available. The 1998 SAP noted the relative lack of information for SWCB, concluding that critical research is needed for SWCB, including: short-term movement, long-distance migration, mating behavior relative to movement (i.e. does mating occur before or after migration). Because of this, in the current state of the art, it is unknown whether IRM strategies designed for ECB (another corn boring pest) will also function optimally for SWCB.

SWCB is an economic pest of corn in some areas (i.e., SW Kansas, SE Colorado, northern Texas, western Oklahoma) and can require regular management. Like ECB, SWCB has 2-4 generations and similar feeding behavior. First generation larvae feed on whorl tissue before tunneling into stalks before pupation, while later generations feed on ear tissue before tunneling into stalks. Females typically mate on the night of emergence and can lay 250-350 eggs (Davis 2000).

Research to investigate the movement patterns of SWCB has been initiated (Buschman et al. 1999). In this mark/recapture study, the following observations were made regarding SWCB from the 1999 data: 1) more males than females were captured at greater distances from the release point (similar to ECB); 2) most recaptures of SWCB were within 100 feet of the release site, although some were also noted at 1200 feet; and 3) the moth movement patterns for ECB and SWCB appear to be similar in most regards. Given these results, it is likely that this part of the IRM strategy (refuge proximity guidelines established for ECB) will also be applicable to SWCB. However, the 1999 results were hampered by low SWCB numbers available for testing and the authors have indicated that this work will continue during the 2000 season.

Research for other secondary pests (e.g., BCW, FAW, SCSB, others) is also lacking and could be useful for specific regions in which these pests may pose an additional concern. However, the 1998 SAP indicated that CEW and SWCB should have the highest priority for biology research among the secondary corn pests.

Based on these characteristics and behavior in agricultural pests, the most commonly used refuge strategy is known as a “block” refuge or “strip” refuge. The NC-205 group has recommended three options for refuge placement relative to Bt corn: blocks planted adjacent to fields, blocks planted within fields, or strips planted within fields (Ostlie et al. 1997). In general, refuges may be deployed as external blocks on the edges or headlands of fields or as strips within the Bt corn field. Research has shown that ECB larvae are capable of moving up to six corn plants within or between rows with the majority of movement occurring within a single row. Later instar (4th and 5th) ECB are more likely to move within rows than between rows (Hellmich 1998). This is a cause for concern because heterozygous (partially resistant) ECB larvae may begin feeding on Bt plants, then move to non-Bt plants (if planted nearby) to complete development, thus defeating the high dose strategy and increasing the risk of resistance. For this reason, seed mixes (refuge created by mixing seed in the hopper) are not typically recommended refuges (Mallet & Porter 1992, Buschman et al. 1997).

Buschman et al. (1997) suggested that the within field refuge is the ideal strategy for an IRM program. Since the ECB larvae tend to move within rows, the authors suggest intact corn rows as an acceptable refuge. Narrow (filling one or two planter boxes with non-Bt corn seed) or wide strips (filling the entire planter with non-Bt seed) may be used as in-field refuges. Data indicate that in-field strips may provide the best opportunity for ECB produced in Bt corn to mate with ECB from non-Bt corn. Since preliminary data suggests that the refuge should be within 100 rows of the Bt corn, Buschman et al. (1997) recommended alternating strips of 96 rows of non-Bt corn and 192 rows of Bt corn. This would result in a 33% refuge that is within 100 rows of the Bt corn.

Currently, in-field strips (planted as complete rows) should extend the full length of the field and include a minimum of six rows planted with non-Bt corn alternating with a Bt corn hybrid. NC-205 has recommended planting six to 12 rows of non-Bt corn when implementing the in-field strip refuge strategy (NC 205 Supplement 1998). The 2000 SAP also agreed that, due to larval movement, wider refuge strips are superior to narrower strips, although planter sizes may restrict strip sizes for some smaller growers (SAP 2001). In-field strips may offer the greatest potential to ensure random mating between susceptible and resistant adults because they can maximize adult genetic mixing. Modeling indicates that strips of at least six rows wide are as effective for ECB IRM as adjacent blocks when a 20% refuge is used (Onstad & Guse 1999). However, strips that are only two rows wide might be as effective as blocks, but may be more risky than either blocks or wider strips given our incomplete understanding of differences in survival between susceptible borers and heterozygotes (Onstad & Gould 1998).

Given the current concerns with larval movement and need for random mating, either external blocks or in-field strips (across the entire field, at least 6 rows wide) are the refuge designs which may provide the most reduction in risk of resistance development. Research indicates that random mating is most likely to occur with in-field strips. However, as noted previously, this IRM strategy presents problems both from a crop damage and farmer compliance perspective.

Further, based on existing scientific belief, refuges must currently be located so that the potential for random mating between susceptible moths (from the refuge) and possible resistant survivors (from the Bt field) is maximized. Therefore, pest flight behavior is a critical variable to consider when discussing refuge proximity. Refuges planted as external blocks should be adjacent or in close proximity to the Bt corn field (Onstad & Gould 1998, Ostlie et al. 1997b). NC-205 initially recommended that refuges should be planted within ½ sections (320 acres) (NC-205 Supplement 1998). Subsequently, the recommendation was revised to specify that non-Bt corn refuges should be placed within ½ mile of the Bt field (¼ mile would be even better) (Ortman 1999).

Hunt et al. (1997) has completed a study which suggests that the majority of ECB do not disperse far from their pupal emergence sites. According to this mark-recapture study, the majority of ECB may not disperse more than 1500 to 2000 feet. A majority (70-98%) of recaptured ECB were trapped within 1500 feet of the release point. However, in an addendum to the 1997 study, the authors caution that the 1500 foot distance does not necessarily represent the maximum dispersal distance for ECB (Hunt et al. 1998a).

Another mark-recapture ECB project was devoted to within-field movement of emerging ECB (in particular unmated females) (Hunt et al. 1998b). Relatively few unmated females were recaptured (10 over the entire experiment), although the majority of those were found within 85 ft of the release point. This suggests that unmated females may not disperse far from the point of pupal eclosion (this was especially true in the irrigated field). In addition, a relatively high proportion of mated females (31%) in irrigated fields were trapped within 10 feet of the release point, suggesting that mating occurred very close to the point of emergence. Both of these observations indicate that many emerging ECB females may not disperse outside of their field of origin. With respect to resistance management and refuge proximity, these results suggest that refuges should be placed in close proximity to Bt corn fields (or as in-field refuge) to increase the chance of random mating (especially for irrigated fields).

In terms of male ECB dispersal, another mark-recapture study by Showers et al. (2001) showed that males dispersing in search of mates may move significant distances (>800 m). However, a greater percentage of males were trapped at closer distances (200 m) to the release point. Based on this research, the authors suggest that, in terms of male movement, the current refuge proximity guidelines of ½ mile should be adequate to ensure mating between susceptible moths and any resistant survivors from the Bt field.

While it is clear that ECB dispersal decreases further from pupal emergence points, the quantitative dispersal behavior of ECB has not been fully determined. However, in terms of optimal refuge placement, under currently-accepted standards, it is considered critical that refuge proximity be selected to maximize the potential for random mating. Based on Hunt et al. data, the closer the refuge is to the Bt corn, the lower the risk of resistance. Since the greatest number of ECB were captured within 1500 feet of the field and most females may mate within ten feet of the field, placing refuges as close to the Bt fields as possible should increase the chance of random mating and decrease the risk of resistance. Currently, the proximity requirement for Bt corn is 1/2 mile (¼ mile in areas where insecticides have been historically used to treat ECB and SWCB) (EPA letter to Bt corn registrants, 1/31/00). The 2000 SAP agreed with this guideline, stating that refuges should be located no further than a half mile (within ¼ mile if possible) from the Bt corn field (SAP 2001).

Of course, each of these refuge options (block, strip, and the like) presents additional challenges in their execution. As noted previously, these methods leave portions of a farmer's field susceptible to insect infestation in order to ensure that susceptible insects develop and are available to mate with any resistant pests in the field. This results in a substantial loss of yield, as currently such refuges must encompass at least 20% of the field. Because of the decreased yield associated with the refuge portion of transgenic pest resistant crops, there are also issues with farmer compliance with the refuge requirements as noted previously.

### Temporal and Spatial Refuge

The use of temporal and spatial mosaics has received some attention as alternate strategies to structured refuge to delay resistance. A temporal refuge, in theory, would manipulate the life cycle of ECB by having the Bt portion of the crop planted at a time in which it would be most attractive to ECB. For example, Bt corn fields would be planted several weeks before conventional corn. Because ECB are thought to preferentially oviposit on taller corn plants, the hope is that the Bt corn will be infested instead of the shorter, less attractive conventional corn. However, there are indications from experts in the field that temporal refuges are an inferior alternative to structured refuges (SAP 1998). Research has shown that planting date cannot be used to accurately predict and manipulate ECB oviposition rates (Calvin et al. 1997, Rice et al. 1997, Ostlie et al. 1997b, Calvin 1998). Local climatic effects on corn phenology make planting date a difficult variable to manipulate to manage ECB. Additional studies will have to be conducted under a broad range of conditions to fully answer this question. In addition, a temporal mosaic may lead to assortive mating in which resistant moths from the Bt crop mate with each other because their developmental time differs from susceptible moths emerging from the refuge (Gould 1994).

Spatial mosaics involve the planting of two separate Bt corn events, with different modes of action. The idea is that insect populations will be exposed to multiple proteins, reducing the likelihood of resistance to any one protein. However, currently-registered products only express one protein and the primary pests of corn (ECB, CEW, SWCB) generally remain on the same plant throughout the larval feeding stages, individual insects will be exposed to only one of the proteins. In the absence of structured refuges producing susceptible insects, resistance may still have the potential to develop in such a system as it would in a single protein monoculture. As a result, the currently-accepted view teaches away from the types of refuge strategies disclosed herein.

It is known that during the growing season CEW move northward from southern overwintering sites to corn-growing regions in the Corn Belt. However, observations of CEW north to south migration (from corn-growing regions to cotton-growing regions) have been noted. Although more research is needed for confirmation, this phenomenon could result in additional exposure to Bt crops and increased selection pressure for CEW resistance. This effect is compounded by the fact that neither Bt cotton or any registered Bt corn event contains a high dose for CEW. As such, it may be necessary to consider additional mitigation measures for CEW.

In considering this issue, the 2000 SAP indicated that CEW refuge is best considered on a regional scale (instead of structured refuge on an individual farm basis), due to the long distance movements typical of this pest (i.e., refuge proximity is not as important for CEW). According to the SAP, a 20% refuge (per farm) would be adequate for CEW, provided the amount of Bt corn in the region does not exceed 50% of the total corn crop. If the regional Bt corn crop exceeds 50%, however, additional structured refuge may be necessary (SAP 2001). However, the SAP did not define what a “region” should be (i.e., county, state, or other division).

Based on the last available acreage data for Bt corn, it should be noted that a number of counties in the Corn Belt exceed the 50% threshold recognized by the 2000 SAP. Because of this, there may be additional risk for CEW resistance. This risk could be mitigated with additional structured refuge in regions with greater than 50% Bt corn. However, additional research will likely be needed to fully determine the risk of CEW north-south movement and appropriate mitigation measures.

### Currently-Accepted Refuge Options

### High Dose Events; MON810, BT11 and TC1507 (Field Corn)

Non-Cotton Regions that do not Spray Insecticides on a Regular Basis

This region encompasses most of the Corn Belt east of the High Plains. The original USDA NC-205 refuge recommendations included a 20-30% untreated structured refuge or a 40% refuge that could be treated with non-Bt insecticides (Ostlie et al. 1997a). In the case of ECB, the primary pest of corn for most of the U.S., it is known that on average less than 10% of growers use insecticide treatment to control this pest (National Center for Food and Agriculture Policy 1999). Because many growers do not regularly treat for ECB, NC-205 modified their position in a May 24, 1999 letter to Dr. Janet Andersen (Director, BPPD). In this letter, NC-205 amended their recommendation to a 20% non-Bt corn refuge that may be treated with insecticides and should be deployed within ½ mile (¼ mile is better) of the Bt corn. Specific recommendations in the letter were: 1) insecticide treatment of refuges should be based on scouting and accepted economic thresholds, 2) treatment should be with a product that does not contain Bt or Cry toxin, 3) records should be kept of treated refuges and shared with the EPA, 4) the potential impact of sprayed refuges should be monitored closely and evaluated annually, and 5) monitoring for resistance should be most intense in higher risk areas, for example where refuges are treated with insecticides (Ortman 1999).

Since most growers do not typically treat field corn with insecticides to control ECB, a refuge of 20% non-Bt corn that may be sprayed with non-Bt insecticides if ECB densities exceed economic thresholds should be viable for the Corn Belt. Refuges can be treated as needed to control lepidopteran stalk-boring insects with non-Bt insecticides or other appropriate IPM practices. Insecticide use should be based on scouting using economic thresholds as part of an IPM program.

Some laboratory studies demonstrate that the Cry2Ab protein alone and the Cry2Ab+Cry1Ac proteins as expressed in Bollgard II produce a functional “high dose” in Bollgard II cotton for control of CBW, TBW, and PBW. These studies will be discussed below. The EPA has previously concluded that a moderate, non-high dose of Cry1Ac is produced in current Bollgard lines to control CBW and a functional high dose of Cry1Ac is produced to control TBW and PBW (USEPA 1998, 2001).

The following table will assist the reader with the acronyms for the insect pests. Note that the table lists the most common pests that are the target of transgenic pest resistance strategies, but the invention is not limited to only these pests.

TABLE 1

| Acronym | Common Name | Scientific Name | Crop |
|---|---|---|---|
| BCW | black cutworm | Agrotis ipsilon (Hufnagel) | corn |
| CBW | cotton | Helicoverpa zea (Boddie) | cotton |
| | bollworm | | |
| CEW | corn earworm | Helicoverpa zea (Boddie) | corn |
| CPB | Colorado | Leptinotarsa decemlineata (Say) | potato |
| | potato beetle | | |
| CSB | common stalk | Papaipema nebris (Guenee) | corn |
| | borer | | |
| ECB | European corn | Ostrinia nubilalis (Huebner) | corn |
| | borer | | |
| FAW | fall armyworm | Spodoptera frugiperda (JE Smith) | corn |
| PBW | pink bollworm | Pectinophora gossypiella | cotton |
| | | (Saunders) | |
| SCSB | southern corn | Diatraea crambidoides (Grote) | corn |
| | stalk borer | | |
| SWCB | southwestern | Diatraea grandiosella (Dyar) | corn |
| | corn borer | | |
| TBW | tobacco | Heliothis virescens (Fabricius) | cotton |
| | budworm | | |

Accordingly, there remains a need for methods for managing pest resistance in a plot of pest resistant crop plants. It would be useful to provide an improved method for the protection of plants, especially corn plants, from feeding damage by pests. It would be particularly useful if such a method would reduce the required application rate of conventional chemical pesticides, and also if it would limit the number of separate field operations that were required for crop planting and cultivation. In addition, it would be useful to have a method of deploying a transgenic refuge required by the regulatory agencies in a field of transgenic crops instead of peripheral to a field of transgenic crops.

### BRIEF SUMMARY OF THE INVENTION

A method for managing pest resistance in a plot of pest resistant crop plants is provided. The method includes cultivating a first pest resistant crop plant in a plot in one planting cycle, and successively cultivating in the next planting cycle a second pest resistant crop plant in the same plot, wherein the first and the second pest resistant crop plants are pesticidal to the same target pest but through a different mode of pesticidal action.

Using the methods of the invention a grower can now plant a corn crop in a plot the planting cycle following the cultivation of corn in the same plot. Prior to the invention, this was not advisable due to the risk of rootworm damage to the crop. Further, since recently there has been rootworm activity in other crops, the methods provide a means of controlling rootworm spread and a resistance management strategy for rootworms.

Corn rootworms of the invention include, for example, Diabrotica virgifera virgifera (LeConte), Diabrotica barberi (Smith and Lawrence), Diabrotica undecimpunctata howardi (Barber), and Diabrotica virgifera zeae (Krysan and Smith). The invention utilizes different modes of pesticidal action. Resistance to rootworms can be introduced into the crop plant by any method known in the art. In some embodiments, the different modes of pesticidal action include toxin binding to different binding sites in the gut membranes of the corn rootworms. Transgenes in the present invention useful against rootworms include, but are not limited to, those encoding the Bt proteins Cry3A, Cry3Bb and Cry34Ab1/Cry35Ab1 protein. Other transgenes appropriate for other pests are discussed herein.

In some embodiments, one or both of the pest resistant crop plants are farther treated with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, carbamates, and combinations thereof. In other embodiments, one or both of the pest resistant crop plants further contain a herbicide resistance gene selected from the group consisting of glyphosate N-acetyltransferase (GAT), 5-enolpyruvylshikimate-3-phosphate synthase (EPSPS), phosphinothricin N-acetyltransferase (PAT), and combinations thereof.

While the invention is described in the context of rootworms, the underlying concepts disclosed herein may also be applied to fields where resistance management is needed in the context other pests, including European corn borer (ECB) (Ostrinia nubilalis), southwestern corn borer (SWCB) (Diatrea grandiosella), corn earworm (CEW) (Helicoverpa zea), western bean cutworm (WBCW) (Loxagrotis albicosta), fall armyworm (FAW) (Spodoptera frugiperda), or black cutworm (BCW) (Agrotis ipsilon). The invention may also be used in combination, such that multiple pests may be controlled in the course of the method, whether by transgenic means or otherwise.

### DETAILED DESCRIPTION OF THE PREFERRED EMBODIMENT

The present invention provides a method directed to managing resistance in a plot of pest resistant crop plants. Specifically, the method includes cultivating a first pest resistant crop plant in a plot in one planting cycle, and successively cultivating in the next planting cycle a second pest resistant crop plant in the same plot, wherein the first and the second pest resistant crop plants are pesticidal to corn rootworm but through a different mode of pesticidal action. It is recognized that the resistance trait can be introduced into the crop plant by transformation (i.e., transgenic) or traditional breeding methods.

By “pesticidal” is intended a toxic effect against a pest (e.g., CRW), and includes activity of either, or both, an externally supplied pesticide and/or an agent that is produced by the crop plants. As used herein, the term “different mode of pesticidal action” includes the pesticidal effects of one or more resistance traits, whether introduced into the crop plants by transformation or traditional breeding methods, such as binding of a pesticidal toxin produced by the crop plants to different binding sites (i.e., different toxin receptors and/or different sites on the same toxin receptor) in the gut membranes of corn rootworms.

As used herein, the term “transgenic pest resistant crop plant” means a plant or progeny thereof (including seeds) derived from a transformed plant cell or protoplast, wherein the plant DNA contains an introduced heterologous DNA molecule, not originally present in a native, non-transgenic plant of the same strain, that confers resistance to one or more corn rootworms. The term refers to the original transformant and progeny of the transformant that include the heterologous DNA. The term also refers to progeny produced by a sexual outcross between the transformant and another variety that includes the heterologous DNA. It is also to be understood that two different transgenic plants can also be mated to produce offspring that contain two or more independently segregating added, heterologous genes. Selfing of appropriate progeny can produce plants that are homozygous for both added, heterologous genes. Back-crossing to a parental plant and out-crossing with a non-transgenic plant are also contemplated, as is vegetative propagation. Descriptions of other breeding methods that are commonly used for different traits and crop plants can be found in one of several references, for example, Fehr (1987), in Breeding Methods for Cultivar Development, ed. J. Wilcox (American Society of Agronomy, Madison, Wis.), each of which is incorporated by reference herein. Breeding methods can also be used to transfer any natural resistance genes into crop plants.

By “plant” is intended major food and fiber crops, including corn, sorghum, wheat, sunflower, cotton, rice, soybean, barley, oil seed rape, and potato, for example. As used herein, the term “corn” means Zea mays or maize and includes all plant varieties that can be bred with corn, including wild maize species. In one embodiment, the disclosed methods are useful for managing resistance in a plot of pest resistant corn, where corn is systematically followed by corn (i.e., continuous corn). In another embodiment, the methods are useful for managing resistance in a plot of first-year pest resistant corn, that is, where corn is followed by another crop (e.g., soybeans), in a two-year rotation cycle. Other rotation cycles are also contemplated in the context of the invention, for example where corn is followed by multiple years of one or more other crops, so as to prevent resistance in other extended diapause pests that may develop over time.

Methods for stably introducing nucleotide sequences into plants and expressing a protein encoded therein are well known in the art. “Introducing” is intended to mean presenting to the plant the nucleotide sequence in such a manner that the sequence gains access to the interior of a cell of the plant. The methods of the invention do not depend on a particular method for introducing a nucleotide sequence into a plant, only that the nucleotide sequence gains access to the interior of the cells of the plant. Transformation protocols as well as protocols for introducing nucleotide sequences into plants may vary depending on the type of plant or plant cell (i.e., monocot or dicot) targeted for transformation.

For example, suitable methods of introducing nucleotide sequences into plants include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840, both of which are herein incorporated by reference), direct gene transfer (Paszkowski et al. (1984) EMBO J. 3:2717-2722), ballistic particle acceleration (see, e.g., U.S. Pat. Nos. 4,945,050; 5,879,918; 5,886,244; and 5,932,782 (each of which is herein incorporated by reference); and Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926), and Lec1 transformation (see, e.g., WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and 5,324,646 (each of which is herein incorporated by reference); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255; Christou and Ford (1995) Annals of Botany 75:407-413 (rice); and Osjoda et al. (1996) Nature Biotechnology 14:745-750 (maize via Agrobacterium tumefaciens).

In other embodiments, the nucleotide sequence may be introduced into plants by contacting the plants with a virus or viral nucleic acids. Generally, such methods involve incorporating the nucleotide sequence within a viral DNA or RNA molecule. It is recognized that the nucleotide sequence may be initially synthesized as part of a viral polyprotein, which later may be processed by proteolysis in vivo or in vitro, to produce the desired recombinant protein. Methods for introducing nucleotide sequences into plants and expressing a protein encoded therein, involving viral DNA or RNA molecules, are known in the art. See, for example, U.S. Pat. Nos. 5,889,191; 5,889,190; 5,866,785; 5,589,367; and 5,316,931 (each of which is herein incorporated by reference); and Porta et al. (1996) Molecular Biotechnology 5:209-221.

Methods are known in the art for the targeted insertion of a nucleotide sequence at a specific location in the plant genome. In one embodiment, the insertion of the nucleotide sequence at a desired genomic location is achieved using a site-specific recombination system. See, for example, WO 99/25821, WO 99/25854, WO 99/25840, WO 99/25855, and WO 99/25853. Briefly, the nucleotide sequence can be contained in a transfer cassette flanked by two non-recombinogenic recombination sites. The transfer cassette is introduced into a plant having stably incorporated into its genome a target site that is flanked by two non-recombinogenic recombination sites that correspond to the sites of the transfer cassette. An appropriate recombinase is provided and the transfer cassette is integrated at the target site. The nucleotide sequence of interest is thereby integrated at a specific chromosomal position in the plant genome.

As used herein, the term “different mode of pesticidal action” includes the pesticidal effects of one or more resistance traits, whether introduced into the crop plants by transformation or traditional breeding methods, such as binding of a pesticidal toxin produced by the crop plants to different binding sites (i.e., different toxin receptors and/or different sites on the same toxin receptor) in the gut membranes of corn rootworms. With regard to modes of pesticidal action, pesticidal compounds bind “competitively” if they have identical binding sites in the pest with no binding sites that one compound will bind that the other will not bind. For example, if compound A uses binding sites 1 and 2 only, and compound B also uses binding sites 1 and 2 only, compounds A and B bind “competitively.” Pesticidal compounds bind “semi-competitively” if they have at least one common binding site in the pest, but also at least one binding site not in common. For example, if compound C uses binding sites 3 and 4, and compound D uses only binding site 3, compounds C and D bind “semi-competitively.” Pesticidal compounds bind “non-competitively” if they have no binding sites in common in the pest. For example, if compound E uses binding sites 5 and 6, and compound F uses binding site 7, compounds E and F bind “non-competitively.”

In some embodiments the different mode of pesticidal action is provided via expression of a heterologous gene derived from a strain of Bacillus thuringiensis, for example, one that encodes an insecticidal δ-endotoxin derived from Bt, where the gene has been stably introduced into the transgenic plants. Such δ-endotoxins are described, for example, in Crickmore et al. (1998) Micro. Mol. Bio. Rev. 62:807-813; U.S. Pat. Nos. 5,691,308; 5,188,960; 6,180,774; 5,689,052 (each of which is herein incorporated by reference); WO 99/24581; and WO 99/31248, and include, for example, genes encoding the Cry proteins Cry1A, Cry1A(a), Cry1A(b), Cry1A(c), Cry1C, Cry1D, Cry1E, Cry1F, Cry2A, Cry3Bb, Cry9C, and variants thereof. It is recognized that variants and fragments of these nucleotide sequences can be used, so long as they encode a Cry polypeptide having pesticidal activity.

The Bt δ-endotoxins are synthesized as protoxins and crystallize as parasporal inclusions. When ingested by an insect pest, the microcrystal structure is dissolved by the alkaline pH of the insect midgut, and the protoxin is cleaved by insect gut proteases to generate the active toxin. The activated Bt toxin binds to receptors in the gut epithelium of the insect, causing membrane lesions and associated swelling and lysis of the insect gut. Insect death results from starvation and septicemia. See, for example, Li et al. (1991) Nature 353:815-821; Aronson (2002) Cell Mol. Life Sci. 59(3):417-425; Schnepf et al. (1998) Micro. Mol. Biol. Rev. 62:775-806.

In other embodiments the heterologous gene is derived from a Bt variant (e.g., Bt var. israelensis), wherein the toxin is unrelated to the Cry family and has a different mode of pesticidal action from the δ-endotoxins. Exemplary toxins include the CytA toxin and the vegetative insecticidal proteins (VIPs). The VIPs (for example, members of the VIP1, VIP2, or VIP3 classes) are secreted proteins that undergo proteolytic processing by midgut insect fluids. They have pesticidal activity against a broad spectrum of Lepidopteran insects. See, for example, U.S. Pat. No. 5,877,012 (herein incorporated by reference).

In further embodiments, the different mode of pesticidal action is provided via expression of a heterologous gene encoding a pesticidal lipase, where the gene has been stably introduced into the transgenic plants. Any nucleotide sequence encoding a lipase polypeptide that has pesticidal activity can be used to practice the methods of the invention. The term “pesticidal lipase” includes any member of the family of lipid acyl hydrolases that has toxic or inhibitory effects on insect pests. Lipases are well known in the art. One class of lipases is the lipid acyl hydrolase class, also known as triacylglycerol acylhydrolases or triacylglycerol lipases (termed EC 3.1.1.3 enzymes under the IUBMB nomenclature system). These enzymes catalyze the hydrolysis reaction: triacylglycerol+H₂O=diacylglycerol+a carboxylate. Lipid acyl hydrolases all share a common, conserved scissile structural region termed the catalytic triad. The catalytic triad consists of a glycine-X amino acid-serine-X amino acid-glycine motif (GxSxG). It has been demonstrated that amino acid substitution in this region abrogates enzymatic activity. The enzymatic action of these lipid acyl hydrolases also correlates with significant insecticidal activity. See, for example, the insecticidal lipases disclosed in U.S. Pat. Nos. 6,657,046 and 5,743,477 (both of which are herein incorporated by reference).

Other pesticidal proteins of use in practicing the methods of the invention include, but are not limited to: binary toxins, such as the Bt crystal proteins of the Cry34 and Cry35 classes (see, e.g., Schnepf et al (2005) Appl. Environ. Microbiol. 71:1765-1774), as well as the cholesterol oxidases from Streptomyces, and pesticidal proteins derived from Xenorhabdus and Photorhabdus bacteria species, Bacillus laterosporous species, and Bacillus sphearicus species. Also contemplated are the use of chimeric (hybrid) toxins (see, e.g., Bosch et al. (1994) Bio/Technology 12:915-918).

The present invention also includes transgenic plants having more than one heterologous gene (i.e., a combination of heterologous genes are stably introduced into the plants). Such transformants can contain transgenes that are derived from the same class of toxin (e.g., more than one δ-endotoxin, more than one pesticidal lipase, more than one binary toxin, and the like), or the transgenes can be derived from different classes of toxins (e.g., a δ-endotoxin in combination with a pesticidal lipase or a binary toxin). For example, a plant having the ability to express an insecticidal δ-endotoxin derived from Bt (such as Cry1F), also has the ability to express at least one other δ-endotoxin that is different from the Cry1F protein, such as, for example, a Cry1A(b) protein. Similarly, a plant having the ability to express an insecticidal δ-endotoxin derived from Bt (such as Cry1F), also has the ability to express a pesticidal lipase, such as, for example, a lipid acyl hydrolase. Likewise, a plant having the ability to express a binary toxin (such as Cry34/35 protein) also has the ability to express at least one other pesticidal protein that is different from the Cry34/35 protein, such as, for example, a δ-endotoxin (e.g., a Cry3Bb protein).

Toxic and inhibitory effects of the Bt toxins and pesticidal lipases include, but are not limited to, stunting of larval growth, killing eggs or larvae, reducing either adult or juvenile feeding on transgenic plants relative to that observed on wild-type plants, and inducing avoidance behavior in an insect as it relates to feeding, nesting, or breeding.

In certain embodiments the nucleotide sequences used in the methods of the present invention can be stacked with any combination of nucleotide sequences of interest in order to create plants with a desired trait. A “trait,” as used herein, refers to the phenotype derived from a particular sequence or groups of sequences. A single expression cassette may contain both a nucleotide encoding a pesticidal protein of interest, and at least one additional gene, such as a gene employed to increase or improve a desired quality of the transgenic plant. Alternatively, the additional gene(s) can be provided on multiple expression cassettes. These stacked combinations can be created by any method including, but not limited to, cross-breeding plants by any conventional or TopCross methodology, or genetic transformation. If the sequences are stacked by genetically transforming the plants, the nucleotide sequences of interest can be combined at any time and in any order. For example, a transgenic plant comprising one or more desired traits (e.g., production of a pesticidal toxin) can be used as the target to introduce further traits by subsequent transformation. The traits can be introduced simultaneously in a co-transformation protocol with the nucleotide sequences of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. It is further recognized that genes can be stacked at a desired genomic location using a site-specific recombination system. See, for example, WO 99/25821, WO 99/25854, WO 99/25840, WO 99/25855, and WO 99/25853.

In practice, certain combinations of the various Bt and other transgenic events described previously are best suited for certain pests, based on the nature of the pesticidal action and the susceptibility of certain pests to certain toxins. For example, some transgenic combinations are particularly suited for use against various types of CRW (including WCRW, NCRW, MCRW, and NCRW). These combinations include Cry34/35 and Cry3A; and Cry34/35 and Cry3B. As previously described, gene stacks may also be used in this context.

Other combinations are also known for other pests. For example, combinations appropriate for use against ECB and/or SWCB include Cry1Ab and Cry1F, Cry1Ab and Cry2, Cry1Ab and Cry9, Cry1Ab and Cry2/Vip3A stack, Cry1Ab and Cry1F/Vip3A stack, Cry1F and Cry2, Cry1F and Cry9, as well as Cry1F and Cry2/Vip3A stack. Combinations appropriate for use against CEW include Cry1Ab and Cry2, Cry1F and Cry2, Cry1Ab and Cry2/Vip3A stack, Cry1Ab and Cry1F/Vip3A stack, as well as Cry1F and Cry2/Vip3A stack. Combinations appropriate for use against FAW, BCW, and/or WBCW include Cry1Ab and Cry2/Vip3A stack, Cry1Ab and Cry1F/Vip3A stack, as well as Cry1F and Cry2/Vip3A stack. Also, these various combinations may be combined in order to provide resistance management to multiple pests.

In other embodiments, the first and/or second pest resistant crop plant is optionally treated with a pesticidal or insecticidal agent. By “pesticidal agent” is intended a chemical pesticide that is supplied externally to the crop plant, or a seed of the crop plant. The term “insecticidal agent” has the same meaning as pesticidal agent, except its use is intended for those instances wherein the pest is an insect. Pesticides suitable for use in the invention include, pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates. Known pesticides within these categories are listed in, for example, The Pesticide Manual, 11th Ed., ed. C. D. S. Tomlin (British Crop Protection Council, Farnham, Surry, UK, 1997).

Insecticides that are oxadiazine derivatives are useful in the subject method. Exemplary oxadizine derivatives for use in the present invention include those that are identified in U.S. Pat. No. 5,852,012 (incorporated herein by reference). Chloronicotinyl insecticides are also useful in the subject method. Exemplary Chloronicotinyls for use in the subject method are described in U.S. Pat. No. 5,952,358 (herein incorporated by reference). Nitroguanidine insecticides are also useful in the present method. Such nitroguanidines can include those described in U.S. Pat. Nos. 5,633,375; 5,034,404 and 5,245,040 (all of which are herein incorporated by reference). Pyrrol, pyrazol and phenyl pyrazol insecticides that are useful in the present method include those that are described in U.S. Pat. No. 5,952,358 (herein incorporated by reference). When an insecticide is described herein, it is to be understood that the description is intended to include salt forms of the insecticide as well as any isomeric and/or tautomeric form of the insecticide that exhibits the same insecticidal activity as the form of the insecticide that is described. The insecticides that are useful in the present method can be of any grade or purity that passes in the trade as such insecticide.

In still other embodiments, the first and/or second pest resistant crop plant is optionally treated with acaricides, nematicides, fungicides, bactericides, herbicides, and combinations thereof.

In further embodiments, the first and/or second pest resistant crop plant further contains a herbicide resistance gene that provides herbicide tolerance, for example, to glyphosate-N-(phosphonomethyl) glycine (including the isopropylamine salt form of such herbicide). Exemplary herbicide resistance genes include glyphosate N-acetyltransferase (GAT) and 5-enolpyruvylshikimate-3-phosphate synthase (EPSPS). Herbicide resistance genes generally code for a modified target protein insensitive to the herbicide or for an enzyme that degrades or detoxifies the herbicide in the plant before it can act. See, DeBlock et al. (1987) EMBO J. 6:2513; DeBlock et al. (1989) Plant Physiol. 91:691; Fromm et al. (1990) BioTechnology 8:833; Gordon-Kamm et al. (1990) Plant Cell 2:603; and Frisch et al. (1995) Plant Mol. Biol. 27:405-9. For example, resistance to glyphosate or sulfonylurea herbicides has been obtained using genes coding for the mutant target enzymes, 5-enolpyruvylshikimate-3-phosphate synthase and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for managing resistance in a plot of pest resistant crop plants comprising the steps of:
a) cultivating a first pest resistant crop plant in a plot in one planting cycle; and
b) cultivating in a second planting cycle a second pest resistant crop plant in the same plot, wherein said first and said second pest resistant crop plants are pesticidal to the same pest but through a different mode of pesticidal action.

2. The method of claim 1, wherein said first and said second pest resistant crop plants are the same species.

3. The method of claim 2, wherein said species is corn.

4. The method of claim 1, wherein said pest is selected from the group consisting of western corn rootworm, northern corn rootworm, Mexican corn rootworm, southern corn rootworm, and combinations thereof.

5. The method of claim 1, wherein said pest is western corn rootworm.

6. The method of claim 1, wherein said first and said second pest resistant crop plants are transgenic pest resistant crop plants.

7. The method of claim 6, wherein said different mode of pesticidal action comprises production of proteins that bind either semi-competitively or non-competitively to different binding sites in the gut membrane of said pest.

8. The method of claim 6, wherein said first transgenic pest resistant crop plant produces a Cry34/35 protein and said second transgenic pest resistant crop plant produces a Cry3A protein.

9. The method of claim 6, wherein said first transgenic pest resistant crop plant produces a Cry34/35 protein and said second transgenic pest resistant crop plant produces a Cry3B protein.

10. The method of claim 6, wherein said first transgenic pest resistant crop plant produces a Cry1A(b) protein and said second transgenic pest resistant crop plant produces a Cry1F protein.

11. The method of claim 6, wherein said first transgenic pest resistant crop plant produces a Cry1A(b) protein and said second transgenic pest resistant crop plant produces a Cry9 protein.

12. The method of claim 1, further comprising treating said first pest resistant crop plant and/or said second pest resistant crop plant with a pesticidal agent.

13. The method of claim 12, wherein said pesticidal agent is selected from the group consisting of insecticides, acaricides, nematicides, fungicides, bactericides, herbicides, and combinations thereof.

14. The method of claim 13, wherein said pesticidal agent is an insecticide.

15. The method of claim 14, wherein said insecticide is selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, carbamates, and combinations thereof.

16. The method of claim 6, wherein said first transgenic pest resistant crop plant or said second transgenic pest resistant crop plant further contains a herbicide resistance gene selected from the group consisting of glyphosate N-acetyltransferase (GAT), 5-enolpyruvylshikimate-3-phosphate synthase (EPSPS), phosphinothricin N-acetyltransferase (PAT), and combinations thereof.
